(19) United States
(12) Patent Application Publication
(10) Pub. No.: US 2025/0255467 A1
(43) Pub. Date: Aug. 14, 2025

(54) ENDOSCOPE FLUID FLOW DIVERTER

(71) Applicant: AdaptivEndo LLC, Louisville, KY (US)

(72) Inventors: Simon M. Furnish, Louisville, KY (US); Walter Stubblefield, Louisville, KY (US); Mark A. Griffin, Louisville, KY (US); Hershel E. Fancher, Georgetown, IN (US)

(21) Appl. No.: 19/195,812

(22) Filed: May 1, 2025

Related U.S. Application Data
(63) Continuation of application No. PCT/US2023/036704, filed on Nov. 2, 2023.
(60) Provisional application No. 63/381,954, filed on Nov. 2, 2022.

(51) Int. Cl. A61B 1/015

(52) U.S. Cl. CPC A61B 1/015

(57) ABSTRACT

An endoscope with a fluid flow diverter is provided. The endoscope includes endoscope body and a tool port cap assembly defining an opening into an instrument path through the body of the endoscope for insertion of an instrument. The tool port cap assembly comprises an upper tool port portion, a lower tool port portion, at least one fluid flow channel, and a diverter seal disposed between the upper tool port portion and the lower tool port portion. The diverter seal is arranged to direct fluid flowing antegrade through the instrument path (i.e., along a distal-to-proximal direction) into the fluid flow channel. Further disclosed are integrated irrigation channels, both primary and auxiliary, incorporated into the endoscope handle.

### CROSS-REFERENCE TO RELATED APPLICATION

This application is a continuation of International Patent Application No. PCT/US2023/036704 filed Nov. 2, 2023 which claims the benefit of U.S. Provisional Application No. 63/381,954 filed Nov. 2, 2022, which are hereby incorporated by reference.

### BACKGROUND OF THE DISCLOSURE

The present disclosure relates generally to endoscopes. In order to minimize the invasiveness of an endoscopic procedure, an endoscope includes a port in which to insert and advance an instrument through an instrument channel and the insertion tube of the endoscope and into the body. Often, fluid backs ups into the instrument channel and leads to splash back of bodily fluids or irrigation into a clinician's face or upper body through the tool port during a procedure. For example, in the field of urology, urine and other fluids sometimes back up and spray into a clinician's face. In order to make the operating room as sterile as possible, as well as to avoid the spraying of fluids on the operating clinician, it is desirable to prevent the splash back of fluids through the tool port.

Thus, there is a need for improvement in this field.

### SUMMARY OF THE DISCLOSURE

Disclosed are fluid flow diverters for a medical device, for example an endoscope. More specifically, although not limited to, small diameter endoscope with two-way or four-way deflection, such as cholangioscopes, ureteroscopes, cystoscopes, and bronchoscopes.

An endoscope assembly may include an endoscope handle and endoscope shaft. The endoscope handle and endoscope shaft define an instrument channel extending therethrough. The endoscope assembly may further include a tool port cap assembly defining an opening for insertion and/or retrieval of an instrument (e.g., a laser fiber, a retrieval basket, a guidewire, and/or any other tool known for use in endoscopes) into the instrument channel (e.g., a fluid path).

The tool port cap assembly may include an upper tool port portion and a lower tool port portion defining at least one fluid flow channel (e.g., defined by the structure of the lower tool port portion). The tool port cap assembly may include a diverter seal disposed between the upper tool port portion and the lower tool port portion. The diverter seal may be compressed between the upper tool port portion and the lower tool port portion. The diverter seal may obstruct at least a portion of the instrument channel to direct fluid that backs up through the instrument channel (i.e., along a distal-to-proximal direction) into the at least one fluid flow channel of the upper tool port portion and/or lower tool port portion.

The at least one fluid flow channel preferably vents to atmospheric pressure (i.e., the pressure in the environment surrounding the endoscope). For example, the at least one fluid flow channel may open to the surrounding environment and/or to an interior portion of the endoscope that is in fluid communication with the surrounding environment (e.g., not sealed). In some examples, the at least one fluid flow channel of the upper tool port portion and/or the lower tool port portion extends away from a proximal end of the tool port cap assembly (i.e., away from the clinician operating the endoscope).

An upper surface of the lower tool port portion may define an opening portion of the instrument channel. The at least one fluid flow channel may be defined in the upper surface of the lower tool port portion. A portion of the at least one fluid flow channel may extend transverse to a longitudinal axis of the lower tool port portion. Further, the diverter seal may contact to the upper surface of the lower tool port portion. The diverter seal may obstruct at least a portion of the instrument channel to divert fluid traveling back through the instrument channel (e.g., along a distal-to-proximal direction) into the at least one fluid flow channel.

The endoscope further includes a seal (e.g., a hemostasis valve, a Tuohy Borst valve, a compression gland, and/or a split septum, just to name a few non-limiting examples) positioned along the instrument path (e.g., a fluid path).

The upper tool port portion and the lower tool port portion may be connected via a snap fit, an interference fit, a friction fit and/or any other suitable connection.

Further disclosed is an endoscope assembly including an endoscope body. The endoscope may further include a tool port cap assembly defining an opening to an instrument path (e.g., a fluid path) for insertion of an instrument (e.g., a tool). The tool port cap assembly may include a seal (e.g., a hemostasis valve, a Tuohy Borst valve, compression gland, and/or a split septum, just to name a few non-limiting examples) with a body defining a portion of the instrument path that extends beyond the body of the seal. The tool port cap assembly may further include a tool port cap defining the opening into the instrument path.

The endoscope may include a diverter seal disposed between the seal and the tool port cap. The diverter seal may contact an upper surface of the seal and/or be spaced from the upper surface of the seal. The diverter seal is preferably arranged to obstruct at least a portion of the instrument path so as to divert fluid traveling back through the instrument path (i.e., along a distal-to-proximal direction) into at least one fluid flow channel. The diverter seal may comprise a hemostasis valve, a Tuohy Borst valve, compression gland, and/or a split septum, just to name a few non-limiting examples.

At least one fluid flow channel communicates with an area between the seal and the diverter seal. For example, an upper surface of the body of the seal may define at least one fluid flow channel, and/or a lower surface of the diverter seal may define at least one fluid flow channel. A portion of the fluid flow channel may have an axis transverse to a longitudinal axis of the fluid path. Preferably, the fluid flow channel vents to the atmosphere.

Further disclosed is an endoscope assembly with an endoscope body. The endoscope assembly may further include a first (e.g., primary) fluid channel defining a first (e.g., primary) fluid flow path at least partially through the body of the endoscope body. The endoscope assembly may also include a second (e.g., auxiliary) fluid channel defining a second (e.g., auxiliary) fluid flow path at least partially through the body of the endoscope. The first and second fluid flow paths may converge into a third fluid flow path.

The first fluid flow path and second fluid flow path may flow into a fitting, for example a manifold fitting. Both of the two fluid flow paths may then exit the manifold fitting (e.g., through a single fluid flow path, the third fluid flow path). The first fluid flow path may be an integrated irrigation channel. The first fluid flow path may be controlled (e.g., partially and/or completely opened and/or closed) via a control (e.g., button) on the endoscope body. The control is discussed in greater detail in US2023/0309802A1 which is herein incorporated by reference in its entirety. A fluid flowing through first fluid flow path may be primarily saline. The proximal end of the second fluid flow path may include a valve (e.g., a syringe activated valve) that can be used for bolus flushing or injecting contrast (e.g., a radiocontrast agent).

The inventive aspects and embodiments discussed below in the following separate paragraphs of the summary may be used independently or in combination with each other.

Further forms, objects, features, aspects, benefits, advantages, and embodiments of the present disclosure will become apparent from a detailed description and drawings provided herewith.

### BRIEF DESCRIPTION OF THE DRAWINGS

FIGS. 1A, 1B, and 1C are perspective views of three embodiments of an endoscope handle with an umbilical cord and an insertion tube.

FIG. 2 is a side view of an upper portion of an endoscope handle (e.g., of FIG. 1A).

FIG. 3 is a cross-sectional view of an endoscope handle (e.g., of FIG. 1A).

FIG. 4 is a side view of a fluid assembly including two irrigation channels and a tool port channel.

FIG. 5 is an exploded side view of a portion of the fluid flow assembly of FIG. 4.

FIG. 6 is a cross-sectional view of the fluid flow assembly of FIG. 4.

FIG. 7 is a side view of a tool port cap assembly.

FIG. 8 is a perspective view of the tool port cap assembly of FIG. 7.

FIG. 9 is an exploded view of the tool port cap assembly of FIG. 7

FIG. 10 is a top view of the tool port cap assembly of FIG. 7.

FIG. 11 is a cross-sectional view taken along line 11-11 in FIG. 10.

### DETAILED DESCRIPTION OF THE DISCLOSURE

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which the disclosure relates. One embodiment of the disclosure is shown in great detail, although it will be apparent to those skilled in the relevant art that some features that are not relevant to the present disclosure may not be shown for the sake of clarity.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

As used herein, “proximal” refers to an end or direction associated with a physician or other treating personnel during a device operation, and “distal” refers to the opposite end (“patient end/treating end”). The drawing figures referred to herein are provided for illustrative purposes only. They should not be construed as limiting the scope of the disclosure defined by the claims, including that they may not necessarily be drawn to scale.

The disclosure is directed to an improved endoscope that incorporates a fluid flow diverter that prevents fluids (e.g., saline, urine, etc.) from spraying back toward the clinician when irrigation is active or when a tool is removed.

One embodiment of the tool cap assembly of an endoscope incorporates a seal into a cap assembly having flow channels to divert antegrade fluid flow through the tool path (i.e., flow along a distal-to-proximal direction) away from the clinician during irrigation or tool removal.

Also disclosed is an integral, primary irrigation channel (e.g., for primarily saline), controlled by a handle mounted control (e.g., button) and an auxiliary irrigation channel, which can have a valve (e.g., a syringe activated valve) that can be used for bolus flushing or injecting contrast (e.g., a radiocontrast agent).

This disclosure could be applied to any small diameter endoscope with two-way or four-way deflection, including, but not limited to: cholangioscopes, ureteroscopes, cystoscopes, and bronchoscopes.

More specifically, disclosed is a tool port cap comprised of an upper and lower portion wherein a diverter seal is positioned between the upper and lower portions of the tool port cap. The upper and/or lower portions of the tool port cap define one or more flow channels that allow any leakage into the area between the seal and the diverter seal (e.g., leakage from the seal) to flow away from the clinician. Further disclosed, integrated irrigation channels, both primary and auxiliary, are also incorporated in this design and disclosed as an improvement over existing endoscopes. The primary irrigation channel places control via, for example, a push button in the clinician's hand rather than control by an assistant using a stop cock. Advantageously, the auxiliary irrigation channel provides for a secondary connection for bolus flushing or injection of contrast solutions without disconnecting the primary irrigation source.

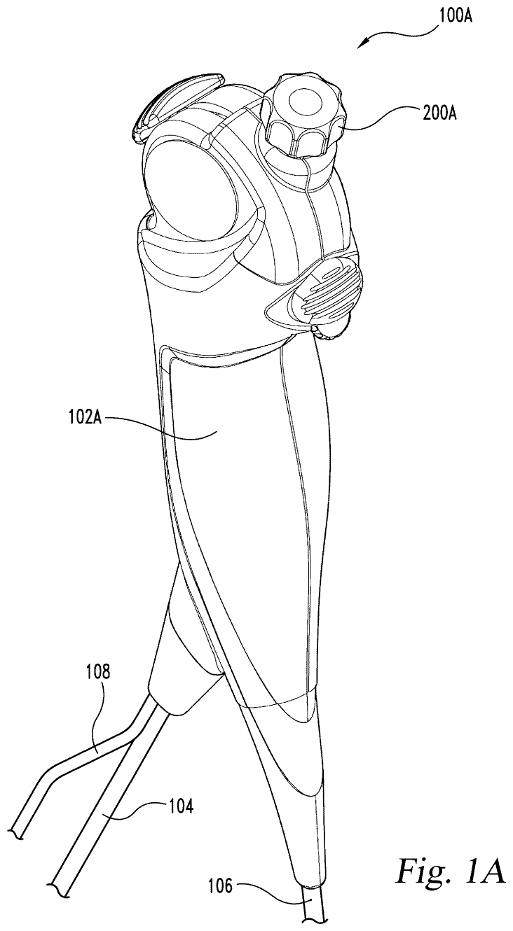

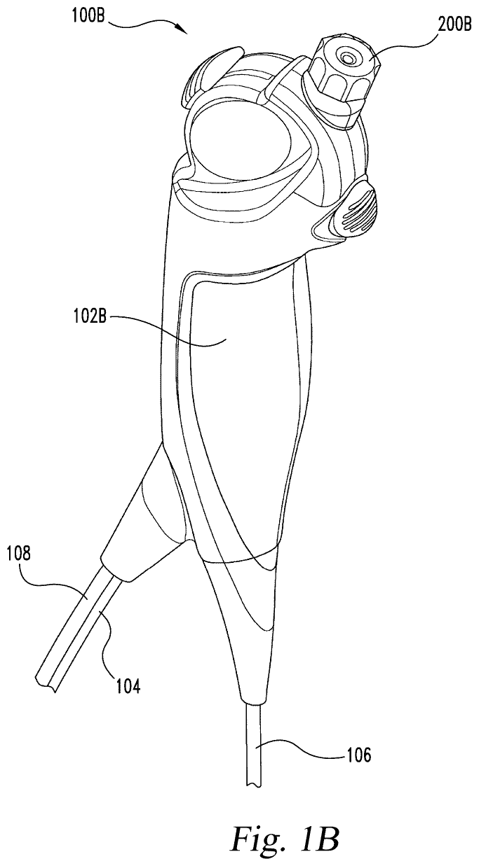

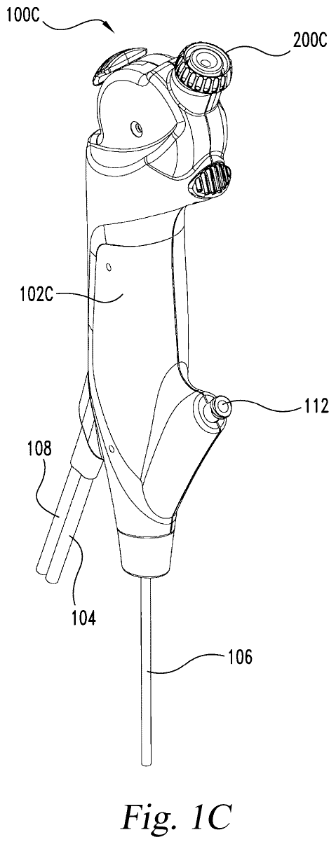

Turning to FIGS. 1A-1C, three endoscope assemblies 100, including endoscope assemblies 100A, 100B and 100C are illustrated. Each endoscope assembly includes a handle body 102, including handle bodies 102A, 102B and 102C. Each endoscope assembly may include a tool port cap assembly 200, including tool port assemblies 200A, 200B, 200C of varying designs. Unless described separately tool port assemblies 200A, 200B, 200C may include the same general structure and function described throughout this disclosure as discussed in relation to tool port cap assembly 200. Additionally, each endoscope assembly may include an umbilical 104 leading to a pump (e.g., a peristaltic pump) for the supply or withdrawal of fluid, an insertion tube 106 for insertion within a patient's body and a second tube 108.

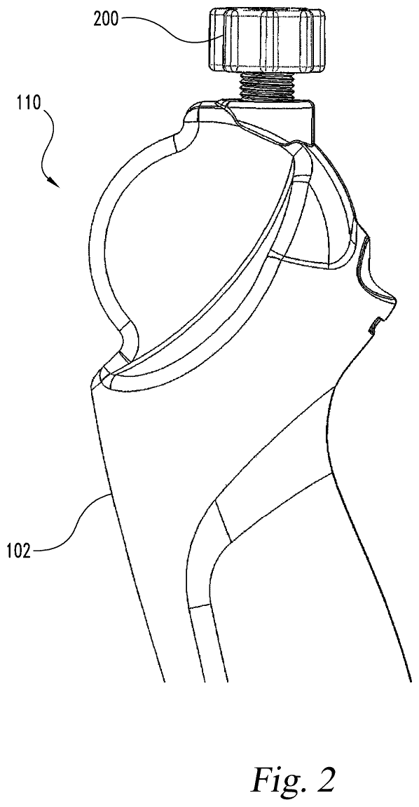

FIG. 2 illustrates an upper portion 110 of an endoscope assembly, including the handle body. As illustrated, the tool port may be positioned on the upper portion of the endoscope assembly. In other embodiments, as illustrated in FIG. 1C a second tool port 112 may be located at a lower and/or more distal location on the handle body.

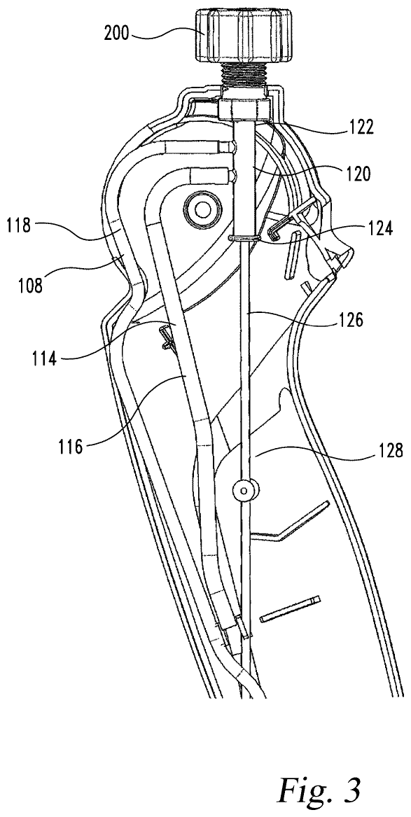

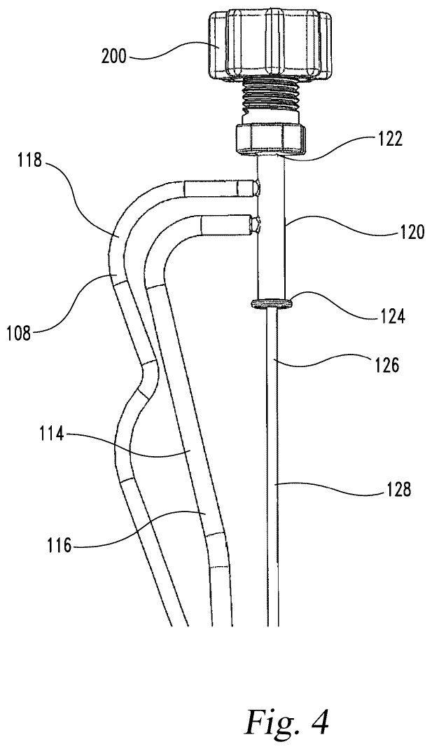

FIGS. 3 and 4 illustrate a first fluid path 114 and a second fluid path 118 having portions in the handle. In some embodiments, the fluid paths may be defined by an interior structure (e.g., wall and/or surface) of the endoscope handle. In other embodiments, the fluid paths may be defined by one or more separate members such as a first tube 116 (e.g., a flexible tube and/or a primary irrigation tube) defining the first fluid path and second tube 108 (e.g., a flexible tube and/or an auxiliary irrigation tube) defining the second fluid path.

In some embodiments, a first tube 116 defining the first fluid path and the second tube 108 defining the second fluid path extend to a fitting 120, for example a manifold fitting as illustrated. The manifold fitting may include a proximal portion 122 and a distal portion 124. In some embodiments, the two fluid paths enter the fitting between the proximal and distal ends of the fitting. In the fitting, the two fluid flow paths may converge (e.g., mix) into a common fluid flow path. For example, the two fluid flow paths may converge into a separate third fluid flow path 126. The fluid flow paths exit the distal end of the fitting via the third fluid flow path 126. The first and second fluid flow paths may converge into a single flow path via any suitable means, for example a t-fitting. In yet other examples, the first and second fluid flow paths may remain separate all the way to the distal end of the insertion tube, such that no convergence of the fluid flow paths occur.

As illustrated and discussed in more detail below, the tool port cap assembly 200 defines an opening into an instrument path, which may include the third fluid flow path. An instrument tube 128 may define the instrument path. The instrument path extends from the opening in the tool port cap assembly to the distal end of the insertion tube to allow various instruments to be inserted and extended through the insertion tube and into a patient's body during a procedure. The instrument path may be synonymous with a tool channel.

In some embodiments, the first fluid flow path is controlled (e.g., partially and/or completely opened and/or closed) via a control (e.g., a button) on the handle body. Such an arrangement is disclosed in greater detail in US2023/0309802A1 which is herein incorporated by reference in its entirety. In other embodiments, the control may be remote from the handle body. Often, the fluid flowing through first fluid flow path is primarily saline. In the same or other embodiments, the proximal end of the second tube (e.g., an auxiliary irrigation tube) includes a valve (e.g., a syringe activated valve, not shown). Advantageously, the auxiliary irrigation channel provides for a secondary connection for bolus flushing or injection of contrast solutions without disconnecting the primary irrigation source.

FIG. 4 provides an illustrative example of the tool port cap assembly, first tube, second tube, and instrument tube without the handle body.

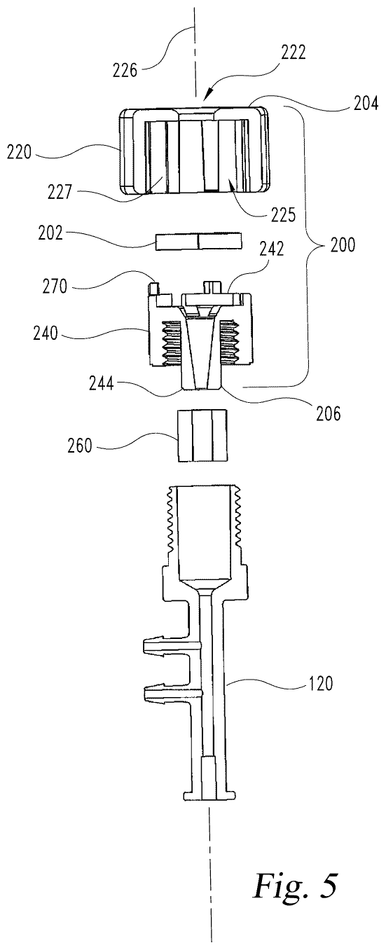

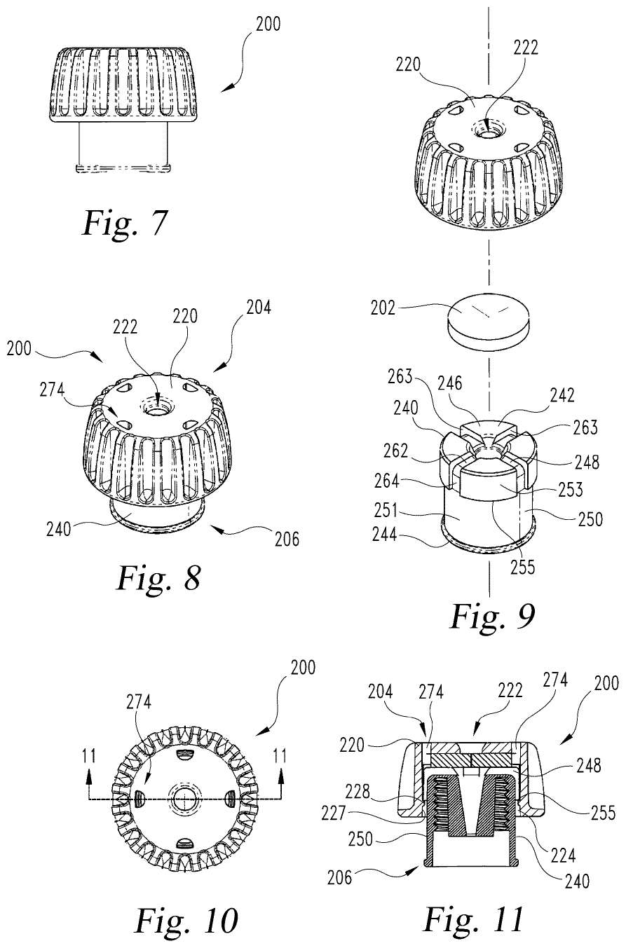

Turning to FIGS. 5-11, the tool port cap assembly is discussed in more detail. The tool port cap assembly 200 may include an upper tool port portion 220, a lower tool port portion 240 and a diverter seal 202. The tool port a cap assembly may include a proximal end 204 and a distal end 206. Tool port cap assembly 200 may define a longitudinal axis 226.

The upper tool port portion 220 may define an opening 222 into the instrument path (e.g., an instrument tube, a third fluid flow path) extending through the body of the endoscope for insertion of an instrument (e.g., a tool). The instrument path extends along the longitudinal axis through the upper tool port portion and the lower tool port portion.

Preferably, the upper tool port portion and/or lower tool port portion define at least one fluid flow channel 224 to divert any unintended fluid flow away from the clinician during irrigation or tool removal. Any number of fluid flow channels 224 may be used. In some exemplary embodiments, four fluid flow channels are used.

The lower tool port portion may include an upper end 242 and a lower end 244, the upper end having an upper surface 246. The upper surface of the lower tool port portion may define an opening into the instrument path. The lower tool portion may include a body 250.

The upper surface 246 may define at least one fluid flow channel portion 248 within the upper surface. At least a portion of the at least one fluid flow channel 248 may extend transverse to a longitudinal axis 226 of the tool port cap assembly. In some embodiments, a first portion 262 of the at least one fluid flow channel may extend transverse to the longitudinal axis of the tool port cap assembly. In some embodiments the at least one fluid flow channel extends perpendicular to the longitudinal axis of the tool port cap assembly. A second portion 264 of the at least one fluid flow channel may then extend downward (e.g., in a direction toward the distal end). In some embodiments the transition from the first portion to the second portion defines a gradual curve. In other embodiments is the transition from the first portion to the second portion defines one or more discrete angles.

One or more fluid flow channels 224 of the upper tool port portion may be in fluid communication with one or more fluid flow channels 248 of the lower tool port portion when the upper tool port portion and the lower tool port portion are connected. Preferably, when connected, the one or more fluid flow channels of the upper tool port portion and the lower tool port portion are in fluid communication to direct the flow of fluid toward the distal end of the tool port cap assembly so that it is directed away from the opening 222 of the upper tool portion.

In some embodiments, the upper tool port portion and the lower tool port portion are connected via a snap fit, an interference fit, and/or a friction fit. It is understood that any other known mechanism for connecting the upper and lower portion may be used alternatively or in combination with these or other connection mechanisms.

In one exemplary embodiment, the upper tool port portion may include a hollow interior 225 defined by an inner surface 227. Inner surface 227 may include a protrusion 228 projecting inwards toward the interior 225 of upper tool port portion 220. In some embodiments, the protrusion extends along a direction transverse to longitudinal axis 226.

The body 250 of lower tool port portion 240 may define portions having different cross-sectional dimensions. For example, a lower portion of body 250 may have an outer surface 251 defining a first cross-sectional dimension (e.g., diameter) while an upper portion may have an outer surface 253 defining a second cross-sectional dimension (e.g., a second diameter) larger than the first cross-sectional dimension. As will be appreciated, the transition from outer surface 251 to outer surface 253 may define a shelf 255. In one example, the shelf 255 extends transverse to longitudinal axis 226. With the lower tool port portion positioned at least partially into interior 225, protrusion 228 may be positioned distally of (e.g., below) and/or contact shelf 255 to retain the upper tool port portion in relation to the lower tool port portion.

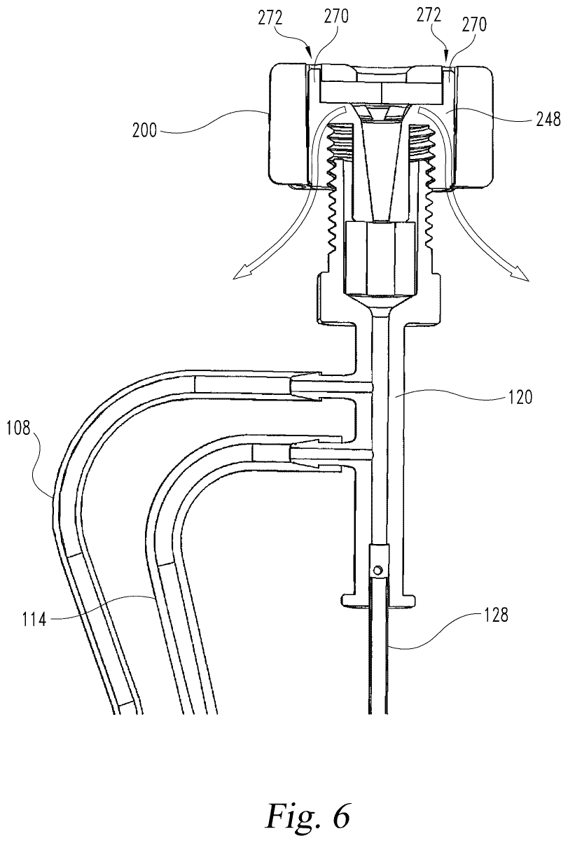

The lower tool port portion may be rotationally limited and/or fixed relative to the upper tool port portion. For example, as shown in FIG. 6, upper surface 246 may include one or more protrusions 270 received by one or more recesses 272 in the upper tool port portion. Advantageously, limiting and/or fixing rotation of the lower tool port portion relative to the upper tool port portion can provide for rotational control of the lower tool port portion by control of the upper tool port portion. Therefore, the tool port cap assembly can be threaded onto the fitting 120 by rotational force applied to the upper tool port portion.

In an alternative embodiment (see FIGS. 9 and 11), upper tool port portion may include one or more downwardly extending protrusions 274 configured to extend into the first portion 262 and/or the second portion 264 of the fluid flow channel. For example, a portion of protrusion 274 may contact a surface 263 defining a side of the first portion 262 so as to limit and/or fix rotation of the lower tool port portion to the upper tool port portion.

In some instances, the upper tool port portion must be translated axially along the longitudinal axis 226 to rotationally engage the lower tool port portion. For example, the upper tool port portion may need to be translated axially towards the lower tool port portion to so that the protrusion of one engages a recess (e.g., channel) of the other to limit and/or fix rotational movement between the two. In some instances, such axial translation of the upper tool port portion relative to the lower tool port portion compresses and/or further compresses the diverter seal.

The diverter seal 202 may be disposed between the upper tool port portion and the lower tool port portion. When the tool port cap assembly is assembled, the diverter seal is preferably compressed between the upper tool port portion and the lower tool port portion. The diverter seal is arranged to resist antegrade flow of fluid (i.e., flow along a distal-to-proximal direction) through the instrument path and the opening in the upper tool port portion. Accordingly, fluid flowing upward from the seal moves, instead, through the one or more fluid flow channels define by the upper tool port portion and/or lower tool port portion.

The diverter seal preferably obstructs at least a portion of the instrument path (e.g., fluid flow path) to divert fluid traveling proximally from the instrument path in the lower tool port portion into the at least one fluid flow channel. In some embodiments, when assembled, the diverter seal contacts the upper surface of the lower tool port portion.

Further, a seal 260 (e.g., a hemostasis valve, a Tuohy Borst valve, a compression gland and/or a split septum) may be associated with the lower tool port portion. The seal is positions downstream of the diverter seal and may be positioned distally (e.g., downstream) of the lower tool port portion along the instrument path. The seal may be compressed between the lower tool port portion and the fitting 120.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the disclosures defined by following claims are desired to be protected. All publications, patents, and patent applications cited in this specification are herein incorporated by reference as if each individual publication, patent, or patent application were specifically and individually indicated to be incorporated by reference and set forth in its entirety herein.

### CLAUSES

The following numbered clauses set out specific embodiments that may be useful in understanding the present disclosure:

Clause 1: An endoscope defining an instrument path having a length extending through the endoscope for receipt of an instrument, the endoscope comprising: a first seal positioned proximally of a second seal along the length of the instrument path and a fluid flow channel in communication with an area of said instrument path between said first seal and said second seal; wherein the fluid flow channel vents to atmospheric pressure.

Clause 2: The endoscope of clause 1, wherein the first seal and the second seal are spaced apart from one another along the instrument path.

Clause 3: The endoscope of any preceding clause, wherein the second seal is a hemostatic seal.

Clause 4: The endoscope of any preceding clause, wherein the second seal is a Tuohy Borst valve.

Clause 5: The endoscope of any preceding clause, wherein the first seal is a split septum seal.

Clause 6: The endoscope of clause 1, wherein the first seal and the second seal are parts of a tool port assembly.

Clause 7: The endoscope of clause 6, wherein the tool port assembly comprises a tool port cap assembly having an upper tool port portion, a lower tool port portion; and wherein the first seal is disposed between the upper tool port portion and the lower tool port portion.

Clause 8: The endoscope of clause 7, wherein the fluid flow channel has at least a portion extending along a direction away from the upper tool port portion.

Clause 9: The endoscope of clause 7 or 8, wherein the fluid flow channel is defined at least in part by the lower tool port portion.

Clause 10: The endoscope of any one of clauses 7-9, wherein the fluid flow channel is defined in the upper surface of the lower tool port portion; and wherein at least a portion of the fluid flow channel extends transverse to a longitudinal axis of the lower tool port portion.

Clause 11: The endoscope of any one of clauses 7-10, wherein the first seal contacts the upper surface of the lower tool port portion; and wherein the first seal obstructs at least a portion of the instrument path to divert fluid traveling along a distal-to-proximal direction along the instrument path towards the first seal into the fluid flow channel.

Clause 12: The endoscope of any one of clauses 7-11, wherein the second seal is positioned distally of said lower tool port portion.

Clause 13: The endoscope of any one of clauses 7-12, wherein rotational movement of the lower tool port portion is limited and/or fixed relative to the upper tool port portion such that the lower tool portion can be rotated by rotation of the upper tool portion.

Clause 14: The endoscope of any one of clauses 7-13, wherein the lower tool port portion has a first portion with a first cross-sectional dimension and a second portion with a second cross-sectional dimension larger than the first cross-sectional dimension; and wherein a transition on an outer surface from the first cross-sectional dimension to the second cross-sectional dimension defines a shelf.

Clause 15: The endoscope of clause 14, wherein the shelf extends transverse to a longitudinal axis of the lower tool port portion.

Clause 16: The endoscope of clause 14 or 15, wherein the lower tool port portion is positioned at least partially in an interior of the upper tool port portion; and wherein a protrusion of the upper tool port portion contacts the shelf to retain the upper tool port portion on the lower tool port portion.

Clause 17: The endoscope of any one of clauses 7-16, wherein the upper tool port portion connects to the lower tool port portion via a snap-fit.

Clause 18: An endoscope comprising: an endoscope body, a first fluid channel defining a first fluid flow path at least partially through the body of the endoscope body; and a second fluid channel defining a second fluid flow path at least partially through the body of the endoscope body, and wherein the first and second fluid flow paths converge into a third fluid flow path; wherein the first fluid flow path is controlled via a control on the endoscope body to selectively open and/or close, partially or completely, the first fluid flow path; wherein the proximal end of the second fluid flow path includes a valve; and wherein the third fluid flow path communicates with an instrument channel for receipt of an instrument therethrough, the instrument channel having a hemostatic seal.

Clause 19: The endoscope of clause 18, wherein the instrument channel defines an instrument path; wherein the endoscope has a diverter seal positioned along the length of the instrument path and spaced proximally of the hemostatic seal and a fluid flow channel in communication with an area of said instrument path between diverter first seal and said hemostatic seal; wherein the fluid flow channel vents to atmospheric pressure.

Clause 20: The endoscope of clause 19, wherein the diverter seal comprises a split septum.

Clause 21: An endoscope comprising: an endoscope body, a tool port assembly defining an opening into an instrument path through the body of the endoscope for insertion of a tool comprising: an upper tool port portion and a lower tool port portion defining a fluid flow channel when the upper tool port portion and the lower tool port portion are connected, and a diverter seal disposed between the upper tool port portion and the lower tool port portion, and wherein the diverter seal directs fluid through the fluid flow channel.

Clause 22: The endoscope of clause 1 wherein a length of the fluid flow channel extends away from the proximal end of the endoscope.

Clause 23: The endoscope of any one of clauses 21-22 wherein the instrument path extends through the upper tool port portion and the lower tool port portion.

Clause 24: The endoscope of any one of clauses 21-23 wherein an upper surface of the lower tool port portion defines an opening into the instrument path.

Clause 25: The endoscope of any one of clauses 21-24 wherein the fluid flow channel is defined in the upper surface of the lower tool port portion, and wherein at least a portion of the fluid flow channel extends transverse to a longitudinal axis of the lower tool port portion.

Clause 26: The endoscope of any one of clauses 21-25 wherein the diverter seal operably contacts the upper surface of the lower tool port portion, wherein the diverter seal obstructs at least a portion of the instrument path to divert fluid traveling through the instrument path into the at least one fluid flow channel.

Clause 27: The endoscope of any one of clauses 21-26 wherein the lower tool port portion includes a split septum.

Clause 28: The endoscope of any one of clauses 21-27 wherein the upper tool port portion defines the opening into the body of the endoscope.

Clause 29: The endoscope of any one of clauses 21-28 further comprising a compressible seal gland positioned distal of the lower tool port portion.

Clause 30: The endoscope of any one of clauses 21-29 wherein the upper tool port portion and the lower tool port portion are connected via a snap fit.

Clause 31: The endoscope of any one of clauses 21-30, wherein the lower tool port portion has a first portion with a first cross-sectional dimension and a second portion with a second cross-sectional dimension larger than the first cross-sectional dimension; and wherein a transition on an outer surface from the first cross-sectional dimension to the second cross-sectional dimension defines a shelf.

Clause 32: The endoscope of any one of clauses 21-31, wherein the shelf extends transverse to a longitudinal axis of the lower tool port portion.

Clause 33: The endoscope of clause 31 or 32, wherein the lower tool port portion is positioned at least partially in an interior of the upper tool port portion; and wherein a protrusion of the upper tool port portion contacts the shelf to retain the upper tool port portion on the lower tool port portion.

Clause 34: An endoscope comprising: an endoscope body, a tool port assembly defining an opening to a fluid path for insertion of a tool comprising: a split septum with a body defining a portion of the fluid path through the body of the split septum, and a tool port cap defining the opening into the fluid path, and wherein the split septum and the tool port cap are connectable to divert fluid away from the opening into the fluid path.

Clause 35: The endoscope of clause 34 further comprising: a diverter seal disposed between the split septum and the tool port cap.

Clause 36: The endoscope of any one of clauses 34 or 35 wherein the diverter seal operably contacts an upper surface of the split septum, wherein the diverter seal covers at least a portion of the fluid path to divert fluid traveling through the fluid path into at least one fluid flow channel defined in the upper surface of the split septum.

Clause 37: The endoscope of any one of clauses 34-36 wherein the split septum defines an opening with an axis transverse to the axis of the fluid path.

Clause 38: The endoscope of any one of clauses 34-37 wherein an upper surface of the body of the split septum defines at least one fluid flow channel, wherein an axis of at least a portion of the fluid flow channel is transverse to a longitudinal axis of the fluid path.

Clause 39: An endoscope comprising: an endoscope body, a first fluid channel defining a first fluid flow path at least partially through the body of the endoscope body; and a second fluid channel defining a second fluid flow path at least partially through the body of the endoscope body, and wherein the first and second fluid flow paths converge into a third fluid flow path.

Clause 40: The endoscope of clause 39 wherein the first fluid flow path and second fluid flow path flow into a mixing fitting, wherein both of the two fluid flow paths exit the mixing fitting through the third fluid flow path.

Clause 41: The endoscope of any one of clauses 39 or 40 wherein the first fluid flow path is an integrated irrigation channel.

Clause 42: The endoscope of any one of clause 39-41; wherein the first fluid flow path is controlled via a control on the endoscope body to selectively open and/or close, partially or completely, the first fluid flow path; wherein the proximal end of the second fluid flow path includes a valve; and wherein the third fluid flow path communicates with an instrument channel for receipt of an instrument therethrough, the instrument channel having a hemostatic seal.

Clause 43: The endoscope of clause 42, wherein the instrument channel defines an instrument path; wherein the endoscope has a diverter seal positioned along the length of the instrument path and spaced proximally of the hemostatic seal and a fluid flow channel in communication with an area of said instrument path between diverter first seal and said hemostatic seal; wherein the fluid flow channel vents to atmospheric pressure.

Clause 44: The endoscope of any one of clauses 39-43 wherein the fluid flowing through first fluid flow path is primarily saline.

Clause 45: The endoscope of any one of clauses 39-44 wherein a proximal end of the second fluid flow path includes a syringe activated valve.

Clause 46: The endoscope of any one of clauses 39-45 wherein the upper tool port portion includes a hollow interior defined by an inner surface, and wherein the inner surface includes a protrusion projecting into the interior of upper tool port portion.

Clause 51: The endoscope of any one of clauses 39-50 wherein an upper surface of the lower tool port portion includes a protrusion, and wherein the protrusion is received by a recess in the upper tool port portion.

Clause 52: The endoscope of any one of clauses 39-51 wherein the upper tool port portion includes at least one protrusion, wherein in a locked configuration the protrusion extends into the fluid flow channel.

Clause 53: The endoscope of any one of clauses 39-52 wherein the fluid flow channel is defined in the upper surface of the lower tool port portion, and wherein the fluid flow channel is defined by a U-shaped depression in the upper surface of the lower tool port portion.

Clause 54: The endoscope of any one of clauses 39-53 wherein the protrusion contacts sidewall of the fluid flow channel.

Clause 55: The endoscope of any one of clauses 39-54 the width of the protrusion is wide enough to that the protrusion directly contacts surfaces on both sides of the fluid flow channel.

Clause 56: The endoscope of any one of clauses 39-55 wherein the width of the protrusion is wide enough so that the protrusion only contacts one surface of the fluid flow channel at a time.

1. An endoscope defining an instrument path having a length extending through the endoscope for receipt of an instrument, the endoscope comprising: a first seal positioned proximally of a second seal along the length of the instrument path and a fluid flow channel in communication with an area of said instrument path between said first seal and said second seal; and wherein the fluid flow channel vents to atmospheric pressure.

2. The endoscope of claim 1, wherein the first seal and the second seal are spaced apart from one another along the instrument path.

3. The endoscope of any preceding claim, wherein the second seal is a hemostatic seal.

4. The endoscope of any preceding claim, wherein the second seal is a Tuohy Borst valve.

5. The endoscope of any preceding claim, wherein the first seal is a split septum seal.

6. The endoscope of claim 1, wherein the first seal and the second seal are parts of a tool port assembly.

7. The endoscope of claim 6, wherein the tool port assembly comprises a tool port cap assembly having an upper tool port portion, a lower tool port portion; and wherein the first seal is disposed between the upper tool port portion and the lower tool port portion.

8. The endoscope of claim 7, wherein the fluid flow channel has at least a portion extending along a direction away from the upper tool port portion.

9. The endoscope of claim 7 or 8, wherein the fluid flow channel is defined at least in part by the lower tool port portion.

10. The endoscope of any one of claims 7-9, wherein the fluid flow channel is defined in the upper surface of the lower tool port portion; and wherein at least a portion of the fluid flow channel extends transverse to a longitudinal axis of the lower tool port portion.

11. The endoscope of any one of claims 7-10, wherein the first seal contacts the upper surface of the lower tool port portion; and wherein the first seal obstructs at least a portion of the instrument path to divert fluid traveling along a distal-to-proximal direction along the instrument path towards the first seal into the fluid flow channel.

12. The endoscope of any one of claims 7-11, wherein the second seal is positioned distally of said lower tool port portion.

13. The endoscope of any one of claims 7-12, wherein rotational movement of the lower tool port portion is limited and/or fixed relative to the upper tool port portion such that the lower tool port portion can be rotated by rotation of the upper tool port portion.

14. The endoscope of any one of claims 7-13, wherein the lower tool port portion has a first portion with a first cross-sectional dimension and a second portion with a second cross-sectional dimension larger than the first cross-sectional dimension; and wherein a transition on an outer surface from the first cross-sectional dimension to the second cross-sectional dimension defines a shelf.

15. The endoscope of claim 14, wherein the shelf extends transverse to a longitudinal axis of the lower tool port portion.

16. The endoscope of claim 14 or 15, wherein the lower tool port portion is positioned at least partially in an interior of the upper tool port portion; and wherein a protrusion of the upper tool port portion contacts the shelf to retain the upper tool port portion on the lower tool port portion.

17. The endoscope of any one of claims 7-16, wherein the upper tool port portion connects to the lower tool port portion via a snap-fit.

18. An endoscope comprising: an endoscope body, a first fluid channel defining a first fluid flow path at least partially through the body of the endoscope body; and a second fluid channel defining a second fluid flow path at least partially through the body of the endoscope body, and wherein the first and second fluid flow paths converge into a third fluid flow path; wherein the first fluid flow path is controlled via a control on the endoscope body to selectively open and/or close, partially or completely, the first fluid flow path; wherein a proximal end of the second fluid flow path includes a valve; and wherein the third fluid flow path communicates with an instrument channel for receipt of an instrument therethrough, the instrument channel having a hemostatic seal.

19. The endoscope of claim 18, wherein the instrument channel defines an instrument path; wherein the endoscope has a diverter seal positioned along a length of the instrument path and spaced proximally of the hemostatic seal and a fluid flow channel in communication with an area of said instrument path between diverter first seal and said hemostatic seal; and wherein the fluid flow channel vents to atmospheric pressure.

20. The endoscope of claim 19, wherein the diverter seal comprises a split septum.
